(19) United States
(12) Patent Application Publication
(10) Pub. No.: US 2026/0272484 A1
(43) Pub. Date: Sep. 17, 2026

(54) INTRAVASCULAR DEVICES AND SYSTEMS WITH ENERGY WAVE DETECTION

(71) Applicant: Cardiovascular Systems, Inc., St. Paul, MN (US)

(72) Inventors: Gregory B. Ingersoll, Minneapolis, MN (US); Joseph P. Higgins, Wayzata, MN (US)

(21) Appl. No.: 19/473,953

(22) PCT Filed: Apr. 12, 2024
(86) PCT No.: PCT/US2024/024296

§ 371 Date: Oct. 9, 2025

Related U.S. Application Data
(60) Provisional application No. 63/458,728, filed on Apr. 12, 2023.

(51) Int. Cl. A61B 17/22; A61B 17/00

(52) U.S. Cl. CPC A61B 17/22022; A61B 17/22029; A61B 2017/00057; A61B 2017/22062; A61B 2017/22065

(57) ABSTRACT

Methods for determining energy wave characteristics within an intravascular lithotripsy device. Methods can include inflating a balloon with a conductive medium to an inflated state, generating a high voltage pulse at a high voltage pulse generator and generating an energy wave within the conductive medium. During at least the generation of the energy wave, emitting light from a laser source at a desired wavelength along a first light guide path to be emitted within the balloon, reflecting emitted light within the balloon by an acousto-optic effect of the energy wave, and sensing reflected light at a photodetector by way of a second light guide path and determining energy wave data from the reflected light.

### CROSS REFERENCE TO RELATED APPLICATIONS

This application claims priority to and the benefit of U.S. Provisional Patent Application No. 63/458,728, filed Apr. 12, 2023, the entire contents of which is incorporated herein by reference in its entirety.

### TECHNICAL FIELD

The present invention is directed to a catheter system for treating a vascular thrombus or calcified lesion or the like utilizing energy waves generated by electrodes within a conductive fluid medium.

### BACKGROUND

Catheter systems having an angioplasty balloon are commonly used to apply a physical force by expansion of the balloon against a calcified lesion within vasculature to force the calcification back into and against the blood vessel wall. Certain such calcified lesions and thrombi are not effectively broken up by the use of an angioplasty balloon.

More recently, catheter systems have been developed that include a balloon similar to an angioplasty balloon that is filled with a conductive liquid medium, such as a saline solution, for expanding the balloon in position at the lesion or thrombus, wherein the catheter system includes one or more pair(s) of electrodes operatively positioned within the conductive liquid medium. The electrodes are pulsed with high voltage direct current so as to create a spark that jumps over a gap between the two electrodes at each pulse. The spark within the conductive medium creates an energy wave that propagates through the liquid medium causing the balloon to physically provide a force against the lesion or thrombus. The energy propagation includes the creation of micro-bubbles that also facilitate the physical force. Such devices are known to provide an energy wave to act against a lesion or thrombus for purposes of breaking up the calcification or clotting.

Current catheter systems include a therapy sequence that includes a maximum number of continuous pulses, followed by a minimum delay time and a hard maximum total pulses associated with a particular catheter. One such product specifies:

| Treatment Frequency | 1 Hz (1 Pulse per | |
|---|---|---|
| | Second) | |
| Maximum Number of Continuous Pulses (1 | 30 | Pulses |
| cycle) | | |
| Minimum Pause Time | 10 | Seconds |
| Maximum Total Pulses Per Catheter | 300 | Pulses |

If a therapy is not yet completed after the maximum total pulse per catheter count, the physician must replace the catheter with the attendant undesirable, cost, delay and distraction.

Intravascular lithotripsy (IVL) devices are available for some calcification patterns. Disposable IVL balloon devices are provided in different designs and sizes for peripheral or coronary indications. All designs utilize a reusable power source such as an IVL generator. One reusable DC generator comprises the following specification:

| Power | 110-240 VAC; 50-60 Hz; Single Phase, 15 A | |
|---|---|---|
| | service | |
| Size | 11″ (28.0 cm) high × 6″ (15.2 cm) wide × | |
| | 11.5″ (29.2 cm) deep | |
| Weight | 15 pounds | (6.8 kg) |
| Output | Proprietary pulse delivery system. Output | |
| | voltage 3000 volts peak, pulse | |
| | frequency 1 Hz | |
| Mobility | Product is designed to be mounted to an IV | |
| | pole | |
| Length | 5 ft | (1.53 m) |

| | |
|---|---|
| Compatibility | Male key distally designed to connect only |
| | to catheter. |
| Operation | Lithotripsy pulsing is activated by pushing a |
| | button on the Connector Cable. |
| Use | Re-usable |

One such disposable device consists of a 0.014-inch guidewire-compatible, fluid-filled balloon angioplasty catheter with two lithotripsy emitters incorporated into the shaft of the 12-mm-long balloon segment. A fluid filled balloon (e.g. a 50/50 saline contrast medium) is inflated to about 4 atm and then electrical pulses are provided to the emitters that create high voltage sparks to provide the therapy. Acoustic waves are created and the calcium is fractured.

### SUMMARY

In accordance with one aspect of the present invention, methods for determining energy wave characteristics within an intravascular lithotripsy device comprising at least one emitter located within an inflatable balloon and electrically connected with a high voltage pulse generator. A method can include inflating the balloon with a conductive medium to an inflated state, generating a high voltage pulse at the high voltage pulse generator and generating an energy wave within the conductive medium, during at least the generation of the energy wave, emitting light from a laser source at a desired wavelength along a first light guide path and within the balloon, reflecting emitted light within the balloon by an acousto-optic effect of the energy wave, and sensing reflected light at a photodetector by way of a second light guide path and determining energy wave data from the reflected light.

Preferably, the high voltage pulse creates an electrical spark between electrodes of an emitter when generated, which spark creates acoustic energy within the conductive medium in the form of a sound wave, the sound wave acting as a reflector of light at specific wavelengths. The energy waves created by the spark or a laser discharge can provide variations (e.g. to the local index of refraction) within the conductive medium that can reflect light at the emitted wavelength of light from the first optical light guide path to be sensed by the photodetector by way of the second light guide path. In particular, the energy wave can generate a discontinuity of the refractive index of the conductive medium that moves through the conductive medium with the energy wave. For comparison, a standing wave would require a continuous source of energy at a particular frequency in a vessel designed with a geometry based on a multiple of wavelengths of the wave. Such a result is inconsistent with an energy impulse from a spark or laser discharge, such as in the case of the present invention. In accordance with the present invention, a moving wave front is created.

The method can also determine energy wave data from reflected light over a plurality of high energy pulses as applied over time. The reflected light sensed at the photodetector can be of a different wavelength than the emitted light.

In another aspect, the method can determine data of an energy wave velocity in a direction moving away from a distal tip of an optical fiber provided as a portion of the second guide path is determined based on a phase change of the reflected light as a function of time.

In accordance with another aspect of the present invention, systems for determining energy wave characteristics within an intravascular lithotripsy device can include an inflatable balloon spaced distally along a catheter of the intravascular lithotripsy device, at least one emitter located within the inflatable balloon and electrically connected with a high voltage pulse generator for generating an energy wave within the conductive medium as a result of a high voltage pulse by the high voltage pulse generator, a laser source for generating light at a desired wavelength along a first light guide path to be emitted within the balloon, and a photodetector located along a second light guide path for sensing reflected light from within the balloon that is reflected as a result of the generation of the energy wave by a high voltage pulse based on an acousto-optic effect of the energy wave.

### BRIEF DESCRIPTION OF THE DRAWINGS

FIG. 1 illustrates a system for providing intravascular lithotripsy according to an aspect of the present invention.

FIG. 2 illustrates an inflated balloon in a vessel for providing intravascular lithotripsy according to an aspect the present invention.

FIG. 3 schematically illustrates an energy wave sensing system of an IVL device in accordance with an aspect of the present invention with a first optical fiber from a laser source for emitting light within a balloon of the IVL device and a second optical fiber for transmitting reflected light from within the balloon to a photosensor.

FIG. 4 is similar to FIG. 3 and illustrates another energy wave sensing system of an IVL device with a shared optical fiber provided from an optical circulator to a distal end of the shared optical fiber within the balloon.

FIG. 5 is a graphical representation comparing energy waves of different strengths as such affects the amount of reflected light.

FIG. 6 is a graphical representation of a manner of determining energy wave speed.

### DETAILED DESCRIPTION

The present invention is directed to IVL devices of the type that include electrodes or lithotripsy emitters that create acoustic waves by arcing discharges between electrode components but may also include devices that create acoustic energy within the balloon via laser energy sources. Examples of such laser systems are described in U.S. Pat. Nos. 11,058,492 and 11,246,569 (the entire contents of which are incorporated by reference). Examples of electrical arc or spark systems are described in U.S. Pat. Nos. 8,728,091, 9,642,673 and 10,850,078 and Published U.S. Pat. Appl. No. 2022-0054194.

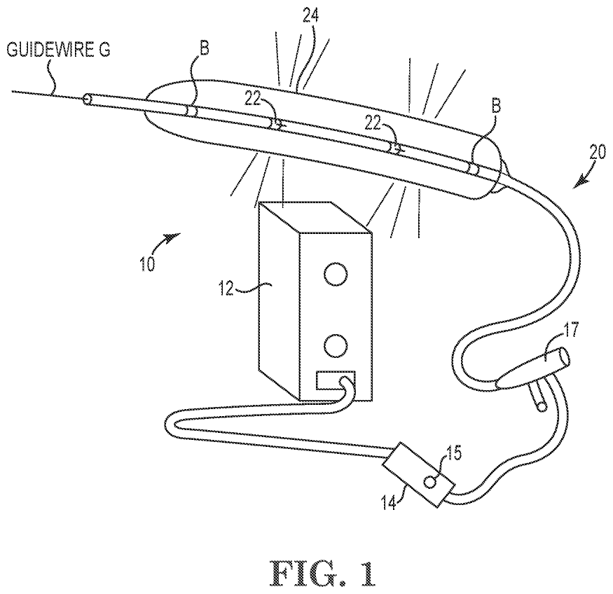

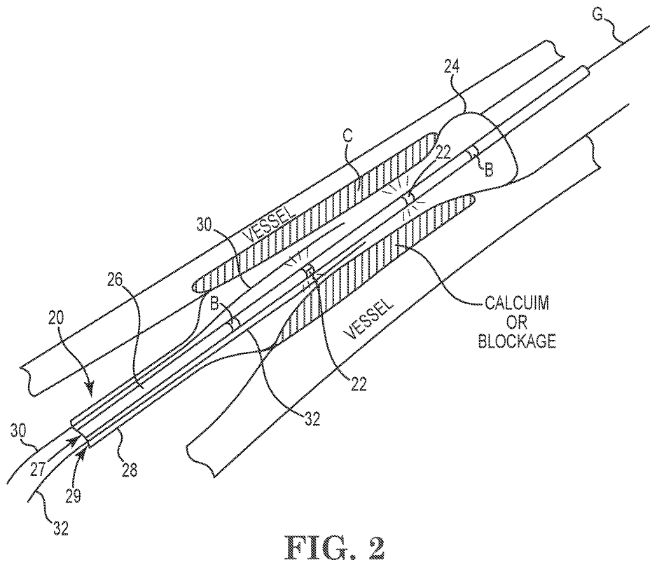

With reference to the Figures, FIGS. 1 and 2 show a system 10 according to the present invention comprising a console or power source 12 (in the form of an electrical generator, but alternatively in the form of a laser system), a handle 14 with therapy delivery control 15 and a catheter 20 with two lithotripsy emitters 22 (shown in the form of a pair of arcing electrodes, but alternatively they could comprise optical or laser emitters), and a fluid filled balloon 24. Optional marker bands B may be provided. The catheter 20 preferably includes a central tube 26 defining a guide wire lumen 27 through which a guide wire G passes for delivering the balloon 24 at the desired location along the guide wire G. A sheath 28 surrounds the central tube 26 and includes a delivery lumen 29 through which saline can be controllably delivered for balloon 24 inflation. The lumen 29 provides a concentric space around the central tube 26 within which electrode wires (not shown) can be run from the control 15 to the emitters 22 among other components in accordance with the present invention and discussed below. The sheath 28 is connected at a proximal end to a hub 17 that can include any number of ports allowing electrode wires to pass into the lumen 29 along with saline for inflation, a guide wire G, and any number of other components as desired.

The balloon 24 may be placed in a deflated position so as to more readily pass through a patient's vasculature to arrive at the scene of calcification. In use, the balloon 24 will be inflated to a common pressure for angioplasty procedures (e.g. 4 atm) and the therapy actuated via the delivery control 15.

FIG. 2 shows the balloon 24 inflated to a therapy delivery state where the lithotripsy emitters 22 may be “fired” to disrupt the vessel calcification C. Optional indicator bands B may be provided to afford visualization and proper positioning by use of known imaging techniques. The balloon 24 can be inflated to an angioplasty pressure (e.g. 4 atm) and therapy can then be delivered. The balloon 24 may naturally expand during or just after the therapy is delivered to clear the vessel for passage of blood.

The control 15 is used to produce one or a series of voltage pulses in accordance with a treatment scheme. A high voltage pulse is provided to one of the emitters 22 comprising a pair of spaced electrodes and, in accordance with the illustrated embodiment, then in series to a second emitter 22 also comprising a pair of spaced electrodes. The high voltage pulse causes a spark across the first electrode pair then across the second electrode pair sequentially within the balloon 24. The somewhat conductive saline solution within the balloon 24 permits the high voltage spark across each electrode pair, thus creating an energy wave that propagates within the balloon toward the vessel calcification.

Any spark created within a balloon 24 that is located within a patient's vasculature will also create a visible, or detectable, light event. Such light event can be detected at wavelengths other than that of visible light. Moreover, it is understood that a visible or detectable light leader can emanate from the ground connected electrode of any electrode pair when a high voltage pulse is initiated on the hot electrode pair prior to the actual spark event. Such a leader is similar to that detectable from conductive objects prior to a lightning strike. Monitoring of detectable light for looking at spark timing is the subject of co-owned pending U.S. provisional patent application No. 63/434,912 filed Dec. 22, 2022, the entire contents of which are incorporated herein by reference.

It is also contemplated that a catheter can be utilized in accordance with the present invention that does not include a balloon. Such a catheter would preferably include a lumen 29 that delivers saline to a controlled volume including the one or more emitters 22. Such a controlled volume can be created by structure of the vasculature of a patient along with the catheter distal end in the area of the emitters. Saline can be provided to fill such a controlled volume or may flow within and from such controlled volume at a controlled flow rate. A partial balloon is also contemplated from which saline fluid flow can weep from an open distal end of a partial balloon. Such a partial or open balloon design can be useful with a forward facing electrode system such as disclosed within pending U.S. provisional patent application No. 63/416,231 filed Oct. 14, 2022, the entire contents of which are incorporated herein by reference. In the case of a catheter 20 having a balloon 24, the controlled volume is provided within the volume of the balloon 24.

As also shown in FIG. 2, a first optical fiber 30 can be run within the lumen 29 to a desired point within the balloon 24 for the purpose of emitting light at a desired wavelength within the balloon 24. A second optical fiber 32 is preferably also run within the lumen 29 to another desired point within the balloon 24 for sensing reflected light emitted from the first optical fiber 30. The distal ends of the first and second fibers 30, 32 are preferably positioned within the balloon 24 for detecting aspects of an energy wave such as is generated by the creation of a high voltage spark at one or more emitters 22, as described above.

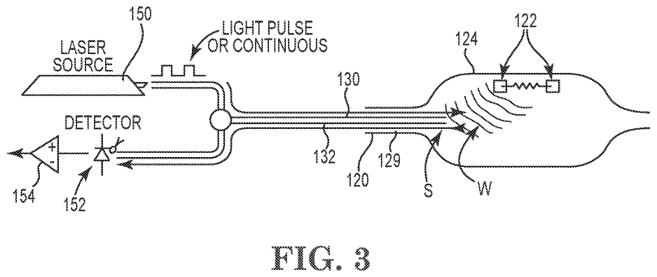

A schematic of a system in accordance with the present invention is shown in FIG. 3. Specifically, a catheter 120 extends to an inflatable balloon 124, in a similar manner as above described. Within a lumen 129, a first optical fiber 130 is run to a select location within the balloon 124 and a second optical fiber 132 is run to another select location within the balloon 124. Electrical wires are also preferably run from a high voltage (e.g. between 100V and 10,000V) pulse generator 154 and within lumen 129 to an emitter 122 for creating a controlled high voltage spark, also as above, for the generation of an energy wave W within a bolus or quantity of conductive solution within the balloon 124. The distal ends of the first and second optical fibers 130, 132 are positioned within the balloon 124 for detecting aspects of such generated energy wave W.

The proximal end of the first optical fiber 130 is preferably optically connected with a laser source 150. It is contemplated that a light source other than a laser can be used, but a laser is preferable in that the light source is provided at a known frequency. The laser source 150 can be a low power laser diode that generates light at a specific wavelength to travel along the optical fiber 130 and to be emitted from the distal end of the first optical fiber 130. The light can be continuous or pulsed, such as coordinated with the high voltage pulses for spark generation at the emitter 122. An advantage of pulsing the light source 150 is that the specific timing of the pulse is known as it can be controlled relative to the high voltage pulse. The light timing can be controlled, for example, to observe different points of time of the high voltage pulse. A continuous light source can allow for observations over time with changes of other use conditions of the system of the present invention.

The proximal end of the second optical fiber 132 is preferably optically connected with a photodetector 152 for detecting light at a specific wavelength, such as light that is emitted from the first optical fiber 130 and reflected within the balloon 124, such as described below. The proximal end of the second optical fiber 132 can be optically connected with an optical detector that preferably provides an amplified signal of the sensed light. Optical fibers are well known as comprising an optical core within which light can propagate that is surrounded by a cladding layer. The cladding layer may further be surrounded by a protective layer. In this embodiment of the present invention, a first light guide path is defined by the optical fiber 130 and a second light guide path is defined by the optical fiber 132.

Light as emitted from the first optical fiber 130 can be reflected based on an acousto-optic effect from the generation of the energy wave W. An acousto-optic effect can act as a wavelength-specific reflector in a similar manner as a fiber Bragg grating. A fiber Bragg grating can be provided within an optical fiber core by creating a periodic variation in the refractive index of the fiber core. Such as fiber Bragg grating reflects particular wavelengths of light and transmits all others. A fiber Bragg grating can be used as an inline optical filter to block certain wavelengths, or it can be used as wavelength-specific reflector.

When acoustic energy is applied to a medium, such as the saline solution within balloon 124, sound waves form within the medium. In the present invention, energy waves W created by the high voltage spark or a laser discharge will provide variations within the medium that reflect light at the emitted wavelength of light from the first optical fiber 130 to be then sensed at the photodetector 152 via the second optical fiber 132. An energy wave W generates a discontinuity of the refractive index of the conductive medium that travels with the wave W. As such, when a wave W is present, some portion of the emitted light from the light source 150 is reflected back to the detector. In any case, some percentage of the light from the light source will be reflected back and can be detected as an observation of the energy wave W. Data determined by the photodetector 152 can include the reflected signal, such as the strength or amplitude of the light, over time and the speed of the energy wave W. By this data, certain characteristics of the energy wave can be determined empirically over time.

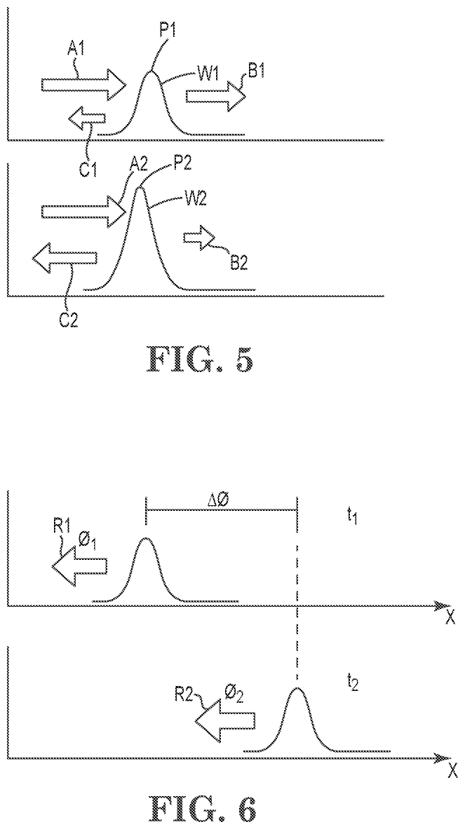

For example, the energy wave W will have an amplitude or strength that is based on the intensity of the spark at any emitter. The higher the strength of the energy wave W, the more impact that will be provided to a lesion. FIG. 5 shows a comparison of two different energy waves W1 and W2 having strengths (shown as wave amplitudes) P1 and P2, respectively. The amplitude of each illustrated wave W1, W2 is shown as the peak P1, P2 of each wave over time. For the sake of illustration, the wave W1 of the upper graph has a lower strength or lower pressure than the wave W2 of the lower graph. Arrow A1 is a representation of the strength of the light source as such is emitted toward the energy wave W1. Arrow B1 represents the amount of emitted light that passes through the wave W1, and arrow C1 represents the amplitude of reflected light that is reflected back from the wave W1. The lower graph illustrates a wave W2 of greater strength than that of W1 (the amplitude P2 of the wave W2 is higher than the amplitude P1 of wave W1). As shown, arrow A2 represents the strength of the light source as emitted toward the energy wave W2. As also shown, arrow A2 can be similar to arrow A1. Arrow B2 represents light passing through the wave W2, the arrow B2 representing less light passing than arrow B1. Arrow C2 represents the amount of reflected light back from wave W2, the arrow C2 representing a larger amount of reflected light than arrow C1.

A wave of greater strength or higher pressure should create a larger change in the index of refraction of the fluid through which the wave passes, which in turn means a higher reflection coefficient at the index change. As such, more reflected light suggests a stronger energy wave W. FIG. 5 represents such a comparison of two waves W1 and W2 of different strengths and the increase in reflected light from a stronger energy wave W.

Another wave aspect that can provide useful information and can be monitored in accordance with the present invention is wave speed. FIG. 6 shows a manner of determining the speed of the wave W optically because the phase (phi) of the reflected light (arrows R1 and R2) changes as a function of time (t₁ to t₂) as the wave moves away from the distal tip of the optical fiber receiving the reflected light. This technique is based on the Doppler effect and optical heterodyne detection (as well-known techniques), but essentially the light is reflecting off of the energy wave that is moving away from the distal tip of the optical fiber receiving the reflected light, so the reflected light will appear to have a lower frequency than the emitted laser light. The difference between the frequencies gives a measure of the wave velocity.

At the photodetector 152, the light can be filtered for the wavelength of the light source 152. It is also contemplated that other light can be detected at other wavelengths by the use of additional photodetectors and filters. For example, light from the spark itself can also be detected for providing data relevant to the spark in addition to data specific to the energy wave W. In this regard, provisional application 63/434,912 filed Dec. 22, 2022 and PCT/US23/85515, filed Dec. 21, 2023 are incorporated herein in their entirety by reference.

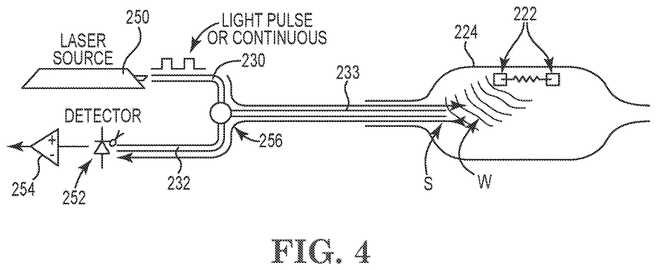

Referring now to FIG. 4, another preferred embodiment of an IVL system of the present invention is schematically illustrated. Instead of having two distinct optical fibers, such as optical fibers 130 and 132 described above, an optical circulator 256 can be provided as part of a first light guide path from the light source 250 to the balloon 224 and as part of a second light guide path from the balloon 224 to the photodetector 252. In this embodiment, a first optical fiber 230 is run from the light source 250 to a first port of the optical circulator 256 and a second optical fiber 232 is run from a third port of the optical circulator to the photodetector. A third optical fiber 233 can then be run from a second port of the optical circulator 256 to a location within the balloon 224. In this way, portions of both the first and second light guide paths can be shared within the third optical fiber 233 from the circulator 256 to within the balloon 224. Such an optical circulator 256 can comprise a three-port circulator where light from the light source 250 passes into the first port of the circulator and out of the second port of the circulator to the balloon 224. Reflected light can pass through the second port of the circulator 256 from the balloon 224 to the photodetector 252 by way of the third port of the circulator. In this system, the circulator 256 separates the source light from the reflected light and controls the first and second light guide paths as above.

It is also contemplated to provide such an acousto-optic determination of energy wave characteristics within a forward facing electrode system such as disclosed within co-owned pending U.S. provisional patent application No. 63/416,231 filed Oct. 14, 2022, and PCT/US23/35025 filed Oct. 12, 2023, the entire contents of which are incorporated herein by reference. With a forward facing or axial firing IVL, a phase change of the emitted light can be detected within the reflected light. For example, emitted light traveling directly away from the first optical fiber 130 in the axial direction, when reflected by an also axial traveling energy wave in either direction can change the wavelength as in the Doppler effect, and as discussed above.

1. A method of determining energy wave characteristics within an intravascular lithotripsy device comprising at least one emitter located within an inflatable balloon and electrically connected with a high voltage pulse generator, the method comprising:
inflating the balloon with a conductive medium to an inflated state;
generating a high voltage pulse at the high voltage pulse generator and generating an energy wave within the conductive medium;
during at least the generation of the energy wave, emitting light from a laser source at a desired wavelength along a first light guide path and within the balloon;
reflecting emitted light within the balloon by an acousto-optic effect of the energy wave; and
sensing reflected light at a photodetector by way of a second light guide path and determining energy wave data from the reflected light.

2. The method of claim 1, wherein the high voltage pulse creates an electrical spark between electrodes of an emitter when generated, which spark creates acoustic energy within the conductive medium in the form of a sound wave, the sound wave acting as a reflector of light at specific wavelengths.

3. The method of claim 2, wherein the acoustic energy is applied as an energy pulse that creates a moving wave front.

4. The method of claim 2, wherein the energy waves created by the spark provide variations within the conductive medium that reflect light at the emitted wavelength of light from the first optical light guide path to be sensed by the photodetector by way of the second light guide path, and thus creating a moving discontinuity.

5. The method of claim 1, wherein the energy wave generates a discontinuity of the refractive index of the conductive medium.

6. The method of claim 1, further comprising determining energy wave data from reflected light over a plurality of high energy pulses as applied over time.

7. The method of claim 1, wherein the reflected light that is sensed at the photodetector is of a different wavelength than the emitted light.

8. The method of claim 1, wherein energy wave velocity in a direction moving away from a distal tip of an optical fiber provided as a portion of the second guide path is determined based on a phase change of the reflected light as a function of time.

9. A method of determining energy wave velocity in a select direction as a characteristic within an intravascular lithotripsy device comprising at least one emitter located within an inflatable balloon and electrically connected with a high voltage pulse generator, the method comprising:
inflating the balloon with a conductive medium to an inflated state;
generating a high voltage pulse at the high voltage pulse generator and generating an energy wave within the conductive medium;
during at least the generation of the energy wave, emitting light from a laser source at a desired wavelength along a first light guide path and within the balloon;
reflecting emitted light within the balloon by an acousto-optic effect of the energy wave; and
sensing reflected light at a photodetector by way of a second light guide path and determining energy wave velocity data in the select direction based upon the reflected light.

10. The method of claim 9, wherein the energy wave velocity data is determined in a direction moving away from a distal tip of an optical fiber provided as a portion of the second light guide path based on a phase change of the reflected light as a function of time.

11. A system of determining energy wave characteristics within an intravascular lithotripsy device comprising:
an inflatable balloon spaced distally along a catheter of the intravascular lithotripsy device from a proximal end of the catheter, the catheter having a lumen for delivering conductive medium to the balloon; and
at least one emitter located within the inflatable balloon and electrically connected with a high voltage pulse generator provided at the proximal end of the catheter, the emitter for generating an energy wave within the conductive medium as a result of a high voltage pulse by the high voltage pulse generator,
the system further comprising:
a laser source for generating light at a desired wavelength along a first light guide path to be emitted within the balloon;
a photodetector located along a second light guide path for sensing reflected light from within the balloon that is reflected as a result of the generation of the energy wave by a high voltage pulse based on an acousto-optic effect of the energy wave.

12. The system of claim 11, wherein the system is controlled to emit light from the laser source at the desired wavelength during the generation of the energy wave.

13. The system of claim 11, wherein the photodetector determines energy wave data from the reflected light.

14. The system of claim 11, wherein the emitter comprises a pair of electrodes so that the high voltage pulse creates an electrical spark between the electrodes, which spark creates acoustic energy within the conductive medium in the form of a sound wave, the sound wave acting as a reflector of light at specific wavelengths.

15. The system of claim 14, wherein the acoustic energy is applied as an energy pulse that creates a moving wave front.

16. The system of claim 11, wherein the plural energy waves created by the spark provide variations within the conductive medium that reflect light at the emitted wavelength of light from the first optical light guide path to be sensed by the photodetector by way of the second light guide path, and thus creating a moving discontinuity.

17. The system of claim 11, wherein the energy wave can generate a discontinuity of the refractive index of the conductive medium.

18. The system of claim 11, further comprising a sensor for determining energy wave data from reflected light over a plurality of high energy pulses as applied over time.

19. The system of claim 11, wherein the reflected light that is sensed at the photodetector can be of a different wavelength than the emitted light.

20. The system of claim 11, wherein energy wave velocity in a direction moving away from a distal tip of an optical fiber provided as a portion of the second guide path can be determined based on a phase change of the reflected light as a function of time.
